(19) United States
(12) Patent Application Publication
(10) Pub. No.: US 2003/0077829 A1
(43) Pub. Date: Apr. 24, 2003

(54) Lipid-based formulations

(75) Inventor: Ian MacLachlan, Vancouver (CA)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Protiva Biotherapeutics Inc.., Burnaby (CA)

(21) Appl. No.: 10/136,707

(22) Filed: Apr. 30, 2002

Related U.S. Application Data
(60) Provisional application No. 60/287,796, filed on Apr. 30, 2001.

(51) Int. Cl. C12N 15/88; A61K 48/00

(52) U.S. Cl. 435/458; 514/44; 435/320.1

(57) ABSTRACT

The present invention provides lipid-based formulations for delivering nucleic acids to a cell, and assays for optimizing the transfection efficiency of such lipid-based formulations.

### CROSS-REFERENCES TO RELATED APPLICATIONS

This application claims the benefit of U.S. patent application Ser. No. 60/287,796, filed Apr. 30, 2001, which is hereby incorporated by reference in its entirety for all purposes.

### BACKGROUND OF THE INVENTION

An effective and safe gene delivery system is required for gene therapy to be clinically useful. Viral vectors are relatively efficient gene delivery systems, but suffer from a variety of limitations, such as the potential for reversion to the wild type as well as immune response concerns. As a result, nonviral gene delivery systems are receiving increasing attention (Worgall, et al., Human Gene Therapy 8:37-44 (1997); Peeters, et al., Human Gene Therapy 7:1693-1699 (1996); Yei, et al., Gene Therapy 1: 192-200 (1994); Hope, et al., Molecular Membrane Biology 15:1-14 (1998)). Plasmid DNA-cationic liposome complexes are currently the most commonly employed nonviral gene delivery vehicles (Felgner, Scientific American 276:102-106 (1997); Chonn, et al., Current Opinion in Biotechnology 6:698-708 (1995)). However, complexes are large, poorly defined systems that are not suited for systemic applications and can elicit considerable toxic side effects (Harrison, et al., Biotechniques 19:816-823 (1995); Huang, et al., Nature Biotechnology 15:620-621 (1997); Templeton, et al., Nature Biotechnology 15:647-652 (1997); Hofland, et al., Pharmaceutical Research 14:742-749 (1997)).

Recent work has shown that plasmid DNA can be encapsulated in small (˜70 nm diameter) “stabilized plasmid-lipid particles” (SPLP) that consist of a single plasmid encapsulated within a bilayer lipid vesicle (Wheeler, et al., Gene Therapy 6:271-281 (1999)). These SPLPs typically contain the “fusogenic” lipid dioleoylphosphatidylethanolamine (DOPE), low levels of cationic lipid, and are stabilized in aqueous media by the presence of a poly(ethylene glycol) (PEG) coating. SPLP have systemic application as they exhibit extended circulation lifetimes following intravenous (i.v.) injection, accumulate preferentially at distal tumor sites due to the enhanced vascular permeability in such regions, and can mediate transgene expression at these tumor sites. The levels of transgene expression observed at the tumor site following i.v. injection of SPLP containing the luciferase marker gene are superior to the levels that can be achieved employing plasmid DNA-cationic liposome complexes (lipoplexes) or naked DNA. Still, improved levels of expression may be required for optimal therapeutic benefit in some applications (see, e.g., Monck, et al., J. Drug Targ. 7:439-452 (2000)).

Thus, there remains a strong need in the art for novel and more efficient methods and compositions for introducing nucleic acids into cells. The present invention addresses this and other needs.

### SUMMARY OF THE INVENTION

In one embodiment, the present invention provides stabilized nucleic acid-lipid particles (SPLPs) and other lipid-based carrier systems containing polyethyleneglycol (PEG)-diacylglycerol (DAG) conjugates, i.e., PEG-DAG conjugates or alternatively DAG-PEG conjugates. In a preferred embodiment, the SPLPs contain a cationic lipid (e.g., DOTMA) a non-cationic lipid (e.g., DSPC), and a PEG-DAG conjugate (e.g. PEG-dilaurylglycerol). Examples of cationic lipids include, but are not limited to, DODAC, DODAP, DODMA, DOTAP, DOTMA, DC-Chol, DMRIE, DSDAC, and DDAB. Suitable non-cationic lipids include, but are not limited to, DSPC, DOPE, DOPC, EPC, cholesterol, and mixtures thereof. Examples of DAG-PEG conjugates include, but are not limited to, a PEG-dilaurylglycerol conjugate (C12), a PEG- dimyristylglycerol (C14) conjugate, a PEG-dipalmitoylglycerol (C16) conjugate and a PEG-disterylglycerol (C18) conjugate. Such SPLPs can used to deliver any of a variety of nucleic acids including, but not limited to, plasmids, antisense oligonucleotides, ribozymes as well as other poly- and oligo-nucleotides.

In a presently preferred embodiment, the nucleic acid encodes a product of interest. a nucleic acid encoding a product of interest (e.g., a restriction endonuclease, a single-chain insulin, a cytokine, etc.). In certain embodiments, the product of interest is a therapeutic product. The therapeutic products can be chosen from a wide variety of compounds including, without limitation, a protein, a nucleic acid, an antisense nucleic acid, ribozymes, tRNA, snRNA, and antigens. In certain embodiments, the therapeutic product encodes a protein, such as those proteins exemplified by the following group: a herpes simplex virus thymidine kinase (HSV-TK), a cytosine deaminase, a xanthine-guaninephosphoribosyl transferase, a p53, purine nucleoside phosphorylase, and a cytochrome P450 2B1. In other embodiments, the therapeutic product encodes a protein selected from the group consisting of: p53, DAP kinase, p16, ARF, APC, neurofibrornin, PTEN, WT1, NF1, and VHL. In still other embodiments, the therapeutic product encodes a protein selected from the group consisting of: angiostatin, endostatin, and VEGF-R2. In still another embodiment, the therapeutic product encodes an Apoptin. The therapeutic products can also be a cytokine, including without limitation: IL-2, IL-3, IL-4, IL-6, IL-7, IL-10, IL-12, IL-15, IFN-α, IFN-β, IFN-γ, TNF-α, GM-CSF, G-CSF, and Flt3-Ligand. Other therapeutic products include, without limitation, antibodies (e.g., a single chain antibody), a peptide hormone, EPO, a single-chain insulin, etc.

In another embodiment, the present invention provides an assay for optimizing the transfection potency of stable nucleic acid-lipid particles based on an endosomal release parameter (ERP). In this assay, an endosomal release parameter (ERP), which is the ratio of the transfection efficiency (measured using a reporter gene, e.g., the luciferase gene) to the uptake efficiency (measured using a detectable label on a component of the nucleic acid-lipid particle), is generated and by comparing the various ERPs of the various nucleic acid-lipid particles, one can optimize the transfection potency. Such assays can be used to optimize not only the SPLPs of the present invention (i.e., those containing PEG-DAG conjugates), but other SPLPs and other cationic lipid containing transfection reagents for both in vitro and in vivo applications.

Other features, objects and advantages of the invention and its preferred embodiments will become apparent from the detailed description which follows.

### BRIEF DESCRIPTION OF THE DRAWINGS

FIG. 1 illustrates the structures of PEG-Diacylglycerols versus PEG-CeramideC₂₀.

FIG. 2 illustrates that clearance studies with LUVs showed that SPLPs containing PEG-DAGs were comparable to SPLPs containing PEG-CeramideC₂₀.

FIG. 3 illustrates that SPLPs containing PEG-DAGs can be formulated via a detergent dialysis method.

FIG. 4 illustrates the in vitro transfection potency of SPLPs containing PEG-DAGs, which were examined in the mouse neuroblastoma cell line, Neuro-2a.

FIG. 5 illustrates the pharmacokinetic properties of SPLPs containing PEG-DAGs.

FIG. 6 illustrates the biodistribution properties of SPLPs containing PEG-DAGs.

FIG. 7 illustrates the luciferase gene expression 24 hrs post IV administration of SPLPs containing PEG-CeramideC₂₀ versus PEG-DAGs in Neuro-2a Tumor Bearing Male A/J Mice.

FIG. 8 illustrates the luciferase gene expression 48 hrs post IV administration of SPLPs containing PEG-CeramideC₂₀ versus PEG-DAGs in Neuro-2a Tumor Bearing Male A/J Mice.

FIG. 9 illustrates the luciferase gene expression 72 hrs post IV administration of SPLPs containing PEG-CeramideC₂₀ versus PEG-DAGs in Neuro-2a Tumor Bearing Male A/J Mice.

FIG. 10 illustrates the ERPs of various SPLPs.

FIG. 11 illustrates the ERPs for SPLPs (A), for SPLPs plus Ca²⁺ (B) and SPLP-CPLs (C).

FIG. 12 illustrates in vitro transfection of Neuro2A cells by SPLP comprising PEG-dilaurylglycerol conjugates.

FIG. 13 illustrates in vitro transfection of Neuro2A cells by SPLP comprising several PEG-diacylglycerol conjugates.

### DETAILED DESCRIPTION OF THE INVENTION AND PREFERRED EMBODIMENTS

In one embodiment, the present invention provides stabilized nucleic acid-lipid particles (SPLPs) and other lipid-based carrier systems containing polyethyleneglycol (PEG)-diacylglycerol (DAG) conjugates, i.e., PEG-DAG conjugates. The lipid-nucleic acid particles of the present invention typically comprise a nucleic acid, a cationic lipid, a non-cationic lipid and a DAG-PEG conjugate. The cationic lipid typically comprises from about 2% to about 60% of the total lipid present in said particle, preferably from about 5% to about 45% of the total lipid present in said particle. In certain preferred embodiments, the cationic lipid comprises from about 5% to about 15% of the total lipid present in said particle. In other preferred embodiments, the cationic lipid comprises from about 40% to about 50% of the total lipid present in said particle. The non-cationic lipid typically comprises from about 5% to about 90% of the total lipid present in said particle, preferably from about 20% to about 85% of the total-lipid present in said particle. The PEG-DAG conjugate typically comprises from 1% to about 20% of the total lipid present in said particle, preferably from 4% to about 15% of the total lipid present in said particle. The nucleic acid-lipid particles of the present invention may further comprise cholesterol. If present, the cholesterol typically comprises from about 10% to about 60% of the total lipid present in said particle, preferably the cholesterol comprises from about 20% to about 45% of the total lipid present in said particle. It will be readily apparent to one of skill in the art that the proportions of the components of the nucleic acid-lipid particles may be varied, e.g., using the ERP assay described in the Example section. For example for systemic delivery, the cationic lipid may comprise from about 5% to about 15% of the total lipid present in said particle and for local or regional delivery, the cationic lipid comprises from about 40% to about 50% of the total lipid present in said particle.

The SPLPs of the present invention typically have a mean diameter of less than about 150 nm and are substantially nontoxic. In addition, the nucleic acids when present in the SPLPs of the present invention are resistant to aqueous solution to degradation with a nuclease. SPLPs and their method of preparation are disclosed in U.S. Pat. No. 5,976,567, U.S. Pat. No. 5,981,501 and PCT Patent Publication No. WO 96/40964, the teachings of all of which are incorporated herein by reference.

Various suitable cationic lipids may be used in the present invention, either alone or in combination with one or more other cationic lipid species or non-cationic lipid species.

Cationic lipids that are useful in the present invention can be any of a number of lipid species which carry a net positive charge at a selected pH, such as physiological pH. Suitable cationic lipids include, but are not limited to, DODAC, DOTMA, DDAB, DOTAP, DOSPA, DOGS, DC-Chol and DMRIE, or combinations thereof. A number of these cationic lipids and related analogs, which are also useful in the present invention, have been described in co-pending U.S. Ser. No. 08/316,399; U.S. Pat. Nos. 5,208,036, 5,264,618, 5,279,833 and 5,283,185, the disclosures of which are incorporated herein by reference. Additionally, a number of commercial preparations of cationic lipids are available and can be used in the present invention. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic liposomes comprising DOGS from Promega Corp., Madison, Wis., USA).

The noncationic lipids used in the present invention can be any of a variety of neutral uncharged, zwitterionic or anionic lipids capable of producing a stable complex. They are preferably neutral, although they can alternatively be positively or negatively charged. Examples of noncationic lipids useful in the present invention include: phospholipid-related materials, such as lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal). Noncationic lipids or sterols such as cholesterol may be present. Additional nonphosphorous containing lipids are, e.g., stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Other lipids such as lysophosphatidylcholine and lysophosphatidylethanolamine may be present. Noncationic lipids also include polyethylene glycol-based polymers such as PEG 2000, PEG 5000 and polyethylene glycol conjugated to phospholipids or to ceramides (referred to as PEG-Cer), as described in co-pending U.S. Ser. No. 08/316,429, incorporated herein by reference.

In preferred embodiments, the noncationic lipids are diacylphosphatidylcholine (e.g., distearoylphosphatidylcholine, dioleoylphosphatidylcholine, dipalmitoylphosphatidylcholine and dilinoleoylphosphatidylcholine), diacylphosphatidylethanolamine (e.g., dioleoylphosphatidylethanolamine and palmitoyloleoylphosphatidylethanolamine), ceramide or sphingomyelin. The acyl groups in these lipids are preferably acyl groups derived from fatty acids having C₁₀-C₂₄ carbon chains. More preferably the acyl groups are lauroyl, myristoyl, palmitoyl, stearoyl or oleoyl. In particularly preferred embodiments, the noncationic lipid will be cholesterol, 1,2-sn-dioleoylphosphatidylethanolamine, or egg sphingomyelin (ESM).

In addition to cationic and non-cationic lipids, the SPLPs of the present invention comprise a diacylglycerol-polyethyleneglycol conjugate, i.e., a DAG-PEG conjugate. The term “diacylglycerol” refers to a compound having 2-fatty acyl chains, R¹ and R², both of which have independently between 2 and 30 carbons bonded to the 1- and 2-position of glycerol by ester linkages. The acyl groups can be saturated or have varying degrees of unsaturation. Diacylglycerols have the following general formula:

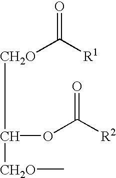

In a presently preferred embodiment, the DAG-PEG conjugate is a di laurylglycerol (C 12)-PEG conjugate, dimyristylglycerol (C14)-PEG conjugate, a dipalmitoylglycerol (C16)-PEG conjugate or a disterylglycerol (C18)-PEG conjugate. Those of skill in the art will readily appreciate that other diacylglycerols can be used in the DAG-PEG conjugates of the present invention.

It has surprisingly been found that PEG-DAG conjugates are particularly useful for SPLP's of the present invention. PEG-DAG conjugates have multiple advantages over PEG-phospholipid derivatives. For example, PEG-phospholipid derivatives have a negative charge on their phosphate group, which leads to multiple disadvantages. First, the negative charge may cause interaction with the cationic lipid in the formulation and, consequently, electrostatic forces that hinder that exchange of the PEG-phospholipid out of the bilayer. Second, the negative charge of the phosphate group neutralizes the cationic charge which is a necessary part of the encapsulation process. To offset the neutralizing effect of the phosphate group, a higher molar percentage of the cationic lipid must be used, thus increasing the toxicity of the formulation. In addition, in contrast to PEG-ceramides, PEG-DAG conjugates are easier to produce and manufacture.

In addition to the foregoing components, the SPLPs of the present invention can further comprise cationic poly(ethylene glycol) (PEG) lipids, or CPLs, that have been designed for insertion into lipid bilayers to impart a positive charge(see, Chen, et al., Bioconj. Chem. 11:433-437 (2000)). Suitable SPLPs and SPLP-CPLs for use in the present invention, and methods of making and using SPLPs and SPLP-CPLs, are disclosed, e.g., in U.S. application Ser. No. 09/553,639, which was filed Apr. 20, 2000, and PCT Patent Application No. CA 00/00451, which was filed Apr. 20, 2000 and which published as WO 00/62813 on Oct. 26, 2000, the teachings of each of which is incorporated herein in its entirety by reference.

In addition to the above components, the SPLPs of the present invention comprise a nucleic acid. While the invention is described herein with reference to the use of plasmids, one of skill in the art will understand that the compositions and methods described herein are equally applicable to other nucleic acids and oligonucleotides. As such, suitable nucleic acids include, but are not limited to, plasmids, antisense oligonucleotides, ribozymes as well as other poly- and oligonucleotides. In preferred embodiments, the nucleic acid encodes a product, e.g., a therapeutic product, of interest.

The product of interest can be useful for commercial purposes, including for therapeutic purposes as a pharmaceutical or diagnostic. Examples of therapeutic products include a protein, a nucleic acid, an antisense nucleic acid, ribozymes, tRNA, snRNA, an antigen, Factor VIII, and Apoptin (Zhuang et al. (1995) Cancer Res. 55(3): 486-489). Suitable classes of gene products include, but are not limited to, cytotoxic/suicide genes, immunomodulators, cell receptor ligands, tumor suppressors, and anti-angiogenic genes. The particular gene selected will depend on the intended purpose or treatment. Examples of such genes of interest are described below and throughout the specification.

Tumor Suppressors

Tumor suppressor genes are genes that are able to inhibit the growth of a cell, particularly tumor cells. Thus, delivery of these genes to tumor cells is useful in the treatment of cancers. Tumor suppressor genes include, but are not limited to, p53 (Lamb et al., Mol. Cell. Biol. 6:1379-1385 (1986), Ewen et al., Science 255:85-87 (1992), Ewen et al. (1991) Cell 66:1155-1164, and Hu et al., EMBO J. 9:1147-1155 (1990)), RB1 (Toguchida et al. (1993) Genomics 17:535-543), WT1 (Hastie, N. D., Curr. Opin. Genet. Dev. 3:408-413 (1993)), NF1 (Trofatter et al., Cell 72:791-800 (1993), Cawthon et al., Cell 62:193-201 (1990)), VHL (Latif et al., Science 260:1317-1320 (1993)), APC (Gorden et al., Cell 66:589-600 (1991)), DAP kinase (see e.g., Diess et al. (1995) Genes Dev. 9: 15-30), p16 (see e.g., Marx (1994) Science 264(5167): 1846), ARF (see e.g., Quelle et al. (1995) Cell 83(6): 993-1000), Neurofibromin (see e.g., Huynh et al. (1992) Neurosci. Lett. 143(1-2): 233-236), and PTEN (see e.g., Li et al. (1997) Science 275(5308): 1943-1947).

Immunomodulator Genes:

Immunomodulator genes are genes that modulate one or more immune responses. Examples of immunomodulator genes include cytokines such as growth factors (e.g., TGF-α., TGF-β, EGF, FGF, IGF, NGF, PDGF, CGF, GM-CSF, G-CSF, SCF, etc.), interleukins (e.g., IL-2, IL-3, IL-4, IL-6, IL-7, IL-10, IL-12, IL-15, IL-20, etc.), interferons (e.g., IFN-α, IFN-β, IFN-γ, etc.), TNF (e.g., TNF-α), and Flt3-Ligand.

Cell Receptor Ligands

Cell receptor ligands include ligands that are able to bind to cell surface receptors (e.g., insulin receptor, EPO receptor, G-protein coupled receptors, receptors with tyrosine kinase activity, cytokine receptors, growth factor receptors, etc.), to modulate (e.g,. inhibit, activate, etc.) the physiological pathway that the receptor is involved in (e.g., glucose level modulation, blood cell development, mitogenesis, etc.). Examples of cell receptor ligands include, but are not limited to, cytokines, growth factors, interleukins, interferons, erythropoietin (EPO), insulin, single-chain insulin (Lee et al. (2000) Nature 408:483-488), glucagon, G-protein coupled receptor ligands, etc.). These cell surface ligands can be useful in the treatment of patients suffering from a disease. For example, a single-chain insulin when expressed under the control of the glucose-responsive hepatocyte-specific L-type pyruvate kinase (LPK) promoter was able to cause the remission of diabetes in streptocozin-induced diabetic rats and autoimmune diabetic mice without side effects (Lee et al. (2000) Nature 408:483-488). This single-chain insulin was created by replacing the 35 amino acid resides of the C-peptide of insulin with a short turn-forming heptapeptide (Gly-Gly-Gly-Pro-Gly-Lys-Arg).

Anti-angiogenic Genes

Anti-angiogenic genes are able to inhibit neovascularization. These genes are particularly useful for treating those cancers in which angiogenesis plays a role in the pathological development of the disease. Examples of anti-angiogenic genes include, but are not limited to, endostatin (see e.g., U.S. Pat. No. 6,174,861), angiostatin (see, e.g., U.S. Pat. No. 5,639,725), and VEGF-R2 (see e.g., Decaussin et al. (1999) J. Pathol. 188(4): 369-737).

Cytotoxic/Suicide Genes

Cytotoxic/suicide genes are those genes that are capable of directly or indirectly killing cells, causing apoptosis, or arresting cells in the cell cycle. Such genes include, but are not limited to, genes for immunotoxins, a herpes simplex virus thymidine kinase (HSV-TK), a cytosine deaminase, a xanthine-guaninephosphoribosyl transferase, a p53, a purine nucleoside phosphorylase, a carboxylesterase, a deoxycytidine kinase, a nitroreductase, a thymidine phosphorylase, and a cytochrome P450 2B 1.

In a gene therapy technique known as gene-delivered enzyme prodrug therapy (“GDEPT”) or, alternatively, the “suicide gene/prodrug” system, agents such as acyclovir and ganciclovir (for thymidine kinase), cyclophosphoamide (for cytochrome P450 2B1), 5-fluorocytosine (for cytosine deaminase), are typically administered systemically in conjunction (e.g., simultaneously or nonsimultaneously, e.g., sequentially) with a expression cassette encoding a suicide gene compositions of the present invention to achieve the desired cytotoxic or cytostatic effect (see, e.g., Moolten, F. L., Cancer Res., 46:5276-5281 (1986)). For a review of the GDEPT system, see, Moolten, F. L., The Internet Book of Gene Therapy, Cancer Therapeutics, Chapter 11 (Sobol, R. E., Scanlon, N.J. (Eds) Appelton & Lange (1995)). In this method, a heterologous gene is delivered to a cell in an expression cassette containing a RNAP promoter, the heterologous gene encoding an enzyme that promotes the metabolism of a first compound to which the cell is less sensitive (i.e., the “prodrug”) into a second compound to which is cell is more sensitive. The prodrug is delivered to the cell either with the gene or after delivery of the gene. The enzyme will process the prodrug into the second compound and respond accordingly. A suitable system proposed by Moolten is the herpes simplex virus-thymidine kinase (HSV-TK) gene and the prodrug ganciclovir. This method has recently been employed using cationic lipid-nucleic aggregates for local delivery (i.e., direct intra-tumoral injection), or regional delivery (i.e., intra-peritoneal) of the TK gene to mouse tumors by Zerrouqui, et al., Can. Gen. Therapy, 3(6):385-392 (1996); Sugaya, et al., Hum. Gen. Ther., 7:223-230 (1996) and Aoki, et al., Hum. Gen. Ther., 8:1105-1113 (1997). Human clinical trials using a GDEPT system employing viral vectors have been proposed (see, Hum. Gene Ther., 8:597-613 (1997), and Hum. Gene Ther., 7:255-267 (1996)) and are underway.

For use with the instant invention, the most preferred therapeutic products are those which are useful in gene-delivered enzyme prodrug therapy (“GDEPT”). Any suicide gene/prodrug combination can be used in accordance with the present invention. Several suicide gene/prodrug combinations suitable for use in the present invention are cited in Sikora, K. in OECD Documents, Gene Delivery Systems at pp. 59-71 (1996), incorporated herein by reference, include, but are not limited to, the following:

| Suicide Gene Product | Less Active ProDrug | Activated Drug |
|---|---|---|
| Herpes simplex virus | ganciclovir(GCV), | phosphorylated |
| type 1 thymidine | acyclovir, | dGTP analogs |
| kinase (HSV-TK) | bromovinyl- | |
| | deoxyuridine, or other | |
| | substrates | |
| Cytosine Deaminase | 5-fluorocytosine | 5-fluorouracil |
| (CD) | | |
| Xanthine-guanine- | 6-thioxanthine (6TX) | 6-thioguano- |
| phosphoribosyl | | sinemonophosphate |
| transferase (XGPRT) | | |
| Purine nucleoside | MeP-dr | 6-methylpurine |
| phosphorylase | | |
| Cytochrome P450 | cyclophosphamide | [cytotoxic |
| 2B1 | | metabolites] |
| Linamarase | amygdalin | cyanide |
| Nitroreductase | CB 1954 | nitrobenzamidine |
| Beta-lactamase | PD | PD mustard |
| Beta-glucuronidase | adria-glu | adriamycin |
| Carboxypeptidase | MTX-alanine | MTX |
| Glucose oxidase | glucose | peroxide |
| Penicillin amidase | adria-PA | adriamycin |
| Superoxide dismutase | XRT | DNA damaging agent |
| Ribonuclease | RNA | cleavage products |

Any prodrug can be used if it is metabolized by the heterologous gene product into a compound to which the cell is more sensitive. Preferably, cells are at least 10-fold more sensitive to the metabolite than the prodrug.

Modifications of the GDEPT system that may be useful with the invention include, 5 for example, the use of a modified TK enzyme construct, wherein the TK gene has been mutated to cause more rapid conversion of prodrug to drug (see, for example, Black, et al., Proc. Natl. Acad. Sci, U.S.A., 93: 3525-3529 (1996)). Alternatively, the TK gene can be delivered in a bicistronic construct with another gene that enhances its effect. For example, to enhance the “bystander effect” also known as the “neighbor effect” (wherein cells in the vicinity of the transfected cell are also killed), the TK gene can be delivered with a gene for a gap junction protein, such as connexin 43. The connexin protein allows diffusion of toxic products of the TK enzyme from one cell into another. The TK/Connexin 43 construct has a CMV promoter operably linked to a TK gene by an internal ribosome entry sequence and a Connexin 43-encoding nucleic acid.

The SPLPs of the present invention, i.e., those SPLPs containing DAG-PEG conjugates, can be made using any of a number of different methods. In one embodiment, the present invention provides lipid-nucleic acid particles produced via hydrophobic nucleic acid-lipid intermediate complexes. The complexes are preferably charge-neutralized. Manipulation of these complexes in either detergent-based or organic solvent-based systems can lead to particle formation in which the nucleic acid is protected.

The present invention provides a method of preparing serum-stable plasmid-lipid particles in which the plasmid or other nucleic acid is encapsulated in a lipid bilayer and is protected from degradation. Additionally, the particles formed in the present invention are preferably neutral or negatively-charged at physiological pH. For in vivo applications, neutral particles are advantageous, while for in vitro applications the particles are more preferably negatively charged. This provides the further advantage of reduced aggregation over the positively-charged liposome formulations in which a nucleic acid can be encapsulated in cationic lipids.

The particles made by the methods of this invention have a size of about 50 to about 150 nm, with a majority of the particles being about 65 to 85 nm. The particles can be formed by either a detergent dialysis method or by a modification of a reverse-phase method which utilizes organic solvents to provide a single phase during mixing of the components. Without intending to be bound by any particular mechanism of formation, a plasmid or other nucleic acid is contacted with a detergent solution of cationic lipids to form a coated plasmid complex. These coated plasmids can aggregate and precipitate. However, the presence of a detergent reduces this aggregation and allows the coated plasmids to react with excess lipids (typically, noncationic lipids) to form particles in which the plasmid or other nucleic acid is encapsulated in a lipid bilayer. The methods described below for the formation of plasmid-lipid particles using organic solvents follow a similar scheme.

In some embodiments, the particles are formed using detergent dialysis. Thus, the present invention provides a method for the preparation of serum-stable plasmid-lipid particles, comprising:

(a) combining a plasmid with cationic lipids in a detergent solution to form a coated plasmid-lipid complex;

(b) contacting noncationic lipids with the coated plasmid-lipid complex to form a detergent solution comprising a plasmid-lipid complex and noncationic lipids; and

(c) dialyzing the detergent solution of step (b) to provide a solution of serum-stable plasmid-lipid particles, wherein the plasmid is encapsulated in a lipid bilayer and the particles are serum-stable and have a size of from about 50 to about 150 nm.

An initial solution of coated plasmid-lipid complexes is formed by combining the plasmid with the cationic lipids in a detergent solution.

In these embodiments, the detergent solution is preferably an aqueous solution of a neutral detergent having a critical micelle concentration of 15-300 mM, more preferably 20-50 mM. Examples of suitable detergents include, for example, N,N′-((octanoylimino)-bis-(trimethylene))-bis-(D-gluconamide) (BIGCHAP); BRIJ 35; Deoxy-BIGCHAP; dodecylpoly(ethylene glycol) ether; Tween 20; Tween 40; Tween 60; Tween 80; Tween 85; Mega 8; Mega 9; Zwittergent® 3-08; Zwittergent® 3-10; Triton X-405; hexyl-, heptyl-, octyl- and nonyl-β-D-glucopyranoside; and heptylthioglucopyranoside; with octyl β-D-glucopyranoside and Tween-20 being the most preferred. The concentration of detergent in the detergent solution is typically about 100 mM to about 2 M, preferably from about 200 mM to about 1.5 M.

The cationic lipids and plasmid will typically be combined to produce a charge ratio (+/−)of about 1:1 to about 20:1, preferably in a ratio of about 1:1 to about 12:1, and more preferably in a ratio of about 2:1 to about 6:1. Additionally, the overall concentration of plasmid in solution will typically be from about 25 μg/mL to about 1 mg/mL, preferably from about 25 μg/mL to about 500 μg/mL, and more preferably from about 100 μg/mL to about 250 μg/mL. The combination of plasmids and cationic lipids in detergent solution is kept, typically at room temperature, for a period of time which is sufficient for the coated complexes to form. Alternatively, the plasmids and cationic lipids can be combined in the detergent solution and warmed to temperatures of up to about 37° C. For plasmids which are particularly sensitive to temperature, the coated complexes can be formed at lower temperatures, typically down to about 4° C.

In a preferred embodiment, the nucleic acid to lipid ratios (mass/mass ratios) in a formed SPLP will range from about 0.01 to about 0.08. The ratio of the starting materials also falls within this range because the purification step typically removes the unencapsulated nucleic acid as well as the empty liposomes. In another preferred embodiment, the SPLP preparation uses about 400 μg nucleic acid per 10 mg total lipid or a nucleic acid to lipid ratio of about 0.01 to about 0.08 and, more preferably, about 0.04, which corresponds to 1.25 mg of total lipid per 50 μg of nucleic acid.

The detergent solution of the coated plasmid-lipid complexes is then contacted with non-cationic lipids to provide a detergent solution of plasmid-lipid complexes and non-cationic lipids. The non-cationic lipids which are useful in this step include, diacylphosphatidylcholine, diacylphosphatidylethanolamine, ceramide, sphingomyelin, cephalin, cardiolipin, and cerebrosides. In preferred embodiments, the non-cationic lipids are diacylphosphatidylcholine, diacylphosphatidylethanolamine, ceramide or sphingomyelin. The acyl groups in these lipids are preferably acyl groups derived from fatty acids having C₁₀-C₂₄ carbon chains. More preferably the acyl groups are lauroyl, myristoyl, palmitoyl, stearoyl or oleoyl. In particularly preferred embodiments, the non-cationic lipid will be 1,2-sn-dioleoylphosphatidylethanolamine (DOPE), palmitoyl oleoyl phosphatidylcholine (POPC), egg phosphatidylcholine (EPC), distearoylphosphatidylcholine (DSPC), cholesterol, or a mixture thereof. In the most preferred embodiments, the nucleic acid-lipid particles will be fusogenic particles with enhanced properties in vivo and the non-cationic lipid will be DSPC or DOPE. As explained above, the nucleic acid-lipid particles of the present invention will further comprise DAG-PEG conjugates. In addition, the nucleic acid-lipid particles of the present invention will further comprise cholesterol.

The amount of non-cationic lipid which is used in the present methods is typically about 0.5 to about 10 mg of total lipids to 50 μg of plasmid. Preferably the amount of total lipid is from about 1 to about 5 mg per 50 μg of plasmid.

Following formation of the detergent solution of nucleic acid-lipid complexes and non-cationic lipids, the detergent is removed, preferably by dialysis. The removal of the detergent results in the formation of a lipid-bilayer which surrounds the nucleic acid providing serum-stable nucleic acid-lipid particles which have a size of from about 50 nm to about 150 nm. The particles thus formed do not aggregate and are optionally sized to achieve a uniform particle size.

The serum-stable nucleic acid-lipid particles can be sized by any of the methods available for sizing liposomes. The sizing may be conducted in order to achieve a desired size range and relatively narrow distribution of particle sizes.

Several techniques are available for sizing the particles to a desired size. One sizing method, used for liposomes and equally applicable to the present particles is described in U.S. Pat. No. 4,737,323, incorporated herein by reference. Sonicating a particle suspension either by bath or probe sonication produces a progressive size reduction down to particles of less than about 50 nm in size. Homogenization is another method which relies on shearing energy to fragment larger particles into smaller ones. In a typical homogenization procedure, particles are recirculated through a standard emulsion homogenizer until selected particle sizes, typically between about 60 and 80 nm, are observed. In both methods, the particle size distribution can be monitored by conventional laser-beam particle size discrimination, or QELS.

Extrusion of the particles through a small-pore polycarbonate membrane or an asymmetric ceramic membrane is also an effective method for reducing particle sizes to a relatively well-defined size distribution. Typically, the suspension is cycled through the membrane one or more times until the desired particle size distribution is achieved. The particles may be extruded through successively smaller-pore membranes, to achieve a gradual reduction in size.

In another group of embodiments, the present invention provides a method for the preparation of serum-stable nucleic acid-lipid particles, comprising:

(a) preparing a mixture comprising cationic lipids and noncationic lipids in an organic solvent;

(b) contacting an aqueous solution of nucleic acid with said mixture in step (a) to provide a clear single phase; and

(c) removing said organic solvent to provide a suspension of plasmid-lipid particles, wherein said plasmid is encapsulated in a lipid bilayer, and said particles are stable in serum and have a size of from about 50 to about 150 nm.

The plasmids (or nucleic acids), cationic lipids and noncationic lipids which are useful in this group of embodiments are as described for the detergent dialysis methods above.

The selection of an organic solvent will typically involve consideration of solvent polarity and the ease with which the solvent can be removed at the later stages of particle formation. The organic solvent, which is also used as a solubilizing agent, is in an amount sufficient to provide a clear single phase mixture of plasmid and lipids. Suitable solvents include, but are not limited to, chloroform, dichloromethane, diethylether, cyclohexane, cyclopentane, benzene, toluene, methanol, or other aliphatic alcohols such as propanol, isopropanol, butanol, tert-butanol, iso-butanol, pentanol and hexanol. Combinations of two or more solvents may also be used in the present invention.

Contacting the nucleic acid with the organic solution of cationic and non-cationic lipids is accomplished by mixing together a first solution of plasmid, which is typically an aqueous solution, and a second organic solution of the lipids. One of skill in the art will understand that this mixing can take place by any number of methods, for example by mechanical means such as by using vortex mixers.

After the nucleic acid has been contacted with the organic solution of lipids, the organic solvent is removed, thus forming an aqueous suspension of serum-stable nucleic acid-lipid particles. The methods used to remove the organic solvent will typically involve evaporation at reduced pressures or blowing a stream of inert gas (e.g., nitrogen or argon) across the mixture.

The serum-stable nucleic acid-lipid particles thus formed will typically be sized from about 50 nm to 150 nm. To achieve further size reduction or homogeneity of size in the particles, sizing can be conducted as described above.

In other embodiments, the methods will further comprise adding nonlipid polycations which are useful to effect the transformation of cells using the present compositions. Examples of suitable nonlipid polycations include, but are limited to, hexadimethrine bromide (sold under the brandname POLYBRENE®, from Aldrich Chemical Co., Milwaukee, Wis., USA) or other salts of heaxadimethrine. Other suitable polycations include, for example, salts of poly-L-ornithine, poly-L-arginine, poly-L-lysine, poly-D-lysine, polyallylamine and polyethyleneimine.

In certain embodiments, the formation of the nucleic acid-lipid particles can be carried out either in a mono-phase system (e.g., a Bligh and Dyer monophase or similar mixture of aqueous and organic solvents) or in a two-phase system with suitable mixing.

When formation of the complexes is carried out in a mono-phase system, the cationic lipids and nucleic acids are each dissolved in a volume of the mono-phase mixture. Combination of the two solutions provides a single mixture in which the complexes form. Alternatively, the complexes can form in two-phase mixtures in which the cationic lipids bind to the nucleic acid (which is present in the aqueous phase), and “pull” it into the organic phase.

In another embodiment, the present invention provides a method for the preparation of nucleic acid-lipid particles, comprising:

(a) contacting nucleic acids with a solution comprising noncationic lipids and a detergent to form a nucleic acid-lipid mixture;

(b) contacting cationic lipids with the nucleic acid-lipid mixture to neutralize a portion of the negative charge of the nucleic acids and form a charge-neutralized mixture of nucleic acids and lipids; and

(c) removing the detergent from the charge-neutralized mixture to provide the lipid-nucleic acid particles in which the nucleic acids are protected from degradation.

In one group of embodiments, the solution of non-cationic lipids and detergent is an aqueous solution. Contacting the nucleic acids with the solution of non-cationic lipids and detergent is typically accomplished by mixing together a first solution of nucleic acids and a second solution of the lipids and detergent. One of skill in the art will understand that this mixing can take place by any number of methods, for example, by mechanical means such as by using vortex mixers. Preferably, the nucleic acid solution is also a detergent solution. The amount of non-cationic lipid which is used in the present method is typically determined based on the amount of cationic lipid used, and is typically of from about 0.2 to 5 times the amount of cationic lipid, preferably from about 0.5 to about 2 times the amount of cationic lipid used.

The nucleic acid-lipid mixture thus formed is contacted with cationic lipids to neutralize a portion of the negative charge which is associated with the nucleic acids (or other polyanionic materials) present. The amount of cationic lipids used will typically be sufficient to neutralize at least 50% of the negative charge of the nucleic acid. Preferably, the negative charge will be at least 70% neutralized, more preferably at least 90% neutralized. Cationic lipids which are useful in the present invention, include, for example, DODAC, DOTMA, DDAB, DOTAP, DC-Chol and DMRIE. These lipids and related analogs have been described in co-pending U.S. Ser. No. 08/316,399; U.S. Pat. Nos. 5,208,036, 5,264,618, 5,279,833 and 5,283,185, the disclosures of which are incorporated herein by reference. Additionally, a number of commercial preparations of cationic lipids are available and can be used in the present invention. These include, for example, LIPOFECTIN® (commercially available cationic liposomes comprising DOTMA and DOPE, from GIBCO/BRL, Grand Island, N.Y., USA); LIPOFECTAMINE® (commercially available cationic liposomes comprising DOSPA and DOPE, from GIBCO/BRL); and TRANSFECTAM® (commercially available cationic lipids comprising DOGS in ethanol from Promega Corp., Madison, Wis., USA).

Contacting the cationic lipids with the nucleic acid-lipid mixture can be accomplished by any of a number of techniques, preferably by mixing together a solution of the cationic lipid and a solution containing the nucleic acid-lipid mixture. Upon mixing the two solutions (or contacting in any other manner), a portion of the negative charge associated with the nucleic acid is neutralized. Nevertheless, the nucleic acid remains in an uncondensed state and acquires hydrophilic characteristics.

After the cationic lipids have been contacted with the nucleic acid-lipid mixture, the detergent (or combination of detergent and organic solvent) is removed, thus forming the lipid-nucleic acid particles. The methods used to remove the detergent will typically involve dialysis. When organic solvents are present, removal is typically accomplished by evaporation at reduced pressures or by blowing a stream of inert gas (e.g., nitrogen or argon) across the mixture.

The particles thus formed will typically be sized from about 100 nm to several microns. To achieve further size reduction or homogeneity of size in the particles, the lipid-nucleic acid particles can be sonicated, filtered or subjected to other sizing techniques which are used in liposomal formulations and are known to those of skill in the art.

In other embodiments, the methods will further comprise adding nonlipid polycations which are useful to effect the lipofection of cells using the present compositions. Examples of suitable nonlipid polycations include, hexadimethrine bromide (sold under the brandname POLYBRENE®, from Aldrich Chemical Co., Milwaukee, Wis., USA) or other salts of hexadimethrine. Other suitable polycations include, for example, salts of poly-L-ornithine, poly-L-arginine, poly-L-lysine, poly-D-lysine, polyallylamine and polyethyleneimine. Addition of these salts is preferably after the particles have been formed.

In another aspect, the present invention provides methods for the preparation of nucleic acid-lipid particles, comprising:

(a) contacting an amount of cationic lipids with nucleic acids in a solution; the solution comprising from about 15-35% water and about 65-85% organic solvent and the amount of cationic lipids being sufficient to produce a +/− charge ratio of from about 0.85 to about 2.0, to provide a hydrophobic lipid-nucleic acid complex;

(b) contacting the hydrophobic, lipid-nucleic acid complex in solution with non-cationic lipids, to provide a nucleic acid-lipid mixture; and

(c) removing the organic solvents from the lipid-nucleic acid mixture to provide lipid-nucleic acid particles in which the nucleic acids are protected from degradation.

The nucleic acids, non-cationic lipids, cationic lipids and organic solvents which are useful in this aspect of the invention are the same as those described for the methods above which used detergents. In one group of embodiments, the solution of step (a) is a mono-phase. In another group of embodiments, the solution of step (a) is two-phase.

In preferred embodiments, the cationic lipids are DODAC, DDAB, DOTMA, DOSPA, DMRIE, DOGS or combinations thereof. In other preferred embodiments, the noncationic lipids are ESM, DOPE, DOPC, DSPC, polyethylene glycol-based polymers (e.g., PEG 2000, PEG 5000 or PEG-modified diacylglycerols), distearoylphosphatidylcholine (DSPC), cholesterol, or combinations thereof. In still other preferred embodiments, the organic solvents are methanol, chloroform, methylene chloride, ethanol, diethyl ether or combinations thereof.

In a particularly preferred embodiment, the nucleic acid is a plasmid; the cationic lipid is DODAC, DDAB, DOTMA, DOSPA, DMRIE, DOGS or combinations thereof; the noncationic lipid is ESM, DOPE, DAG-PEGs, distearoylphosphatidylcholine (DSPC), cholesterol, or combinations thereof (e.g. DSPC and DAG-PEGs); and the organic solvent is methanol, chloroform, methylene chloride, ethanol, diethyl ether or combinations thereof.

As above, contacting the nucleic acids with the cationic lipids is typically accomplished by mixing together a first solution of nucleic acids and a second solution of the lipids, preferably by mechanical means such as by using vortex mixers. The resulting mixture contains complexes as described above. These complexes are then converted to particles by the addition of non-cationic lipids and the removal of the organic solvent. The addition of the non-cationic lipids is typically accomplished by simply adding a solution of the non-cationic lipids to the mixture containing the complexes. A reverse addition can also be used. Subsequent removal of organic solvents can be accomplished by methods known to those of skill in the art and also described above.

The amount of non-cationic lipids which is used in this aspect of the invention is typically an amount of from about 0.2 to about 15 times the amount (on a mole basis) of cationic lipids which was used to provide the charge-neutralized lipid-nucleic acid complex. Preferably, the amount is from about 0.5 to about 9 times the amount of cationic lipids used.

In yet another aspect, the present invention provides lipid-nucleic acid particles which are prepared by the methods described above. In these embodiments, the lipid-nucleic acid particles are either net charge neutral or carry an overall charge which provides the particles with greater gene lipofection activity. Preferably, the nucleic acid component of the particles is a nucleic acid which encodes a desired protein or blocks the production of an undesired protein. In preferred embodiments, the nucleic acid is a plasmid, the noncationic lipid is egg sphingomyelin and the cationic lipid is DODAC. In particularly preferred embodiments, the nucleic acid is a plasmid, the noncationic lipid is a mixture of DSPC and cholesterol, and the cationic lipid is DOTMA. In other particularly preferred embodiments, the noncationic lipid may further comprise cholesterol.

A variety of general methods for making SPLP-CPLs (CPL-containing SPLPs) are discussed herein. Two general techniques include “post-insertion” technique, that is, insertion of a CPL into for example, a preformed SPLP, and the “standard” technique, wherein the CPL is included in the lipid mixture during for example, the SPLP formation steps. The post-insertion technique results in SPLPs having CPLs mainly in the external face of the SPLP bilayer membrane, whereas standard techniques provide SPLPs having CPLs on both internal and external faces.

In particular, “post-insertion” involves forming SPLPs (by any method), and incubating the pre-formed SPLPs in the presence of CPL under appropriate conditions (preferably 2-3 hours at 60° C.). Between 60-80% of the CPL can be inserted into the external leaflet of the recipient vesicle, giving final concentrations up to about 5 to 10 mol % (relative to total lipid). The method is especially useful for vesicles made from phospholipids (which can contain cholesterol) and also for vesicles containing PEG-lipids (such as PEG-DAGs).

In an example of a “standard” technique, the CPL-SPLPs of the present invention can be formed by extrusion. In this embodiment, all of the lipids including the CPL, are co-dissolved in chloroform, which is then removed under nitrogen followed by high vacuum. The lipid mixture is hydrated in an appropriate buffer, and extruded through two polycarbonate filters with a pore size of 100 nm. The resulting SPLPs contain CPL on both of the internal and external faces. In yet another standard technique, the formation of CPL-SPLPs can be accomplished using a detergent dialysis or ethanol dialysis method, for example, as discussed in U.S. Pat. Nos. 5,976,567 and 5,981,501, both of which are incorporated herein by reference.

The nucleic acid-lipid particles of the present invention can be administered either alone or in mixture with a physiologically-acceptable carrier (such as physiological saline or phosphate buffer) selected in accordance with the route of administration and standard pharmaceutical practice. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc.

The pharmaceutical carrier is generally added following particle formation. Thus, after the particle is formed, the particle can be diluted into pharmaceutically acceptable carriers such as normal saline.

The concentration of particles in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, particles composed of irritating lipids may be diluted to low concentrations to lessen inflammation at the site of administration.

As described above, the nucleic acid-lipid particles of the present invention comprise DAG-PEG conjugates. It is often desirable to include other components that act in a manner similar to the DAG-PEG conjugates and that serve to prevent particle aggregation and to provide a means for increasing circulation lifetime and increasing the delivery of the nucleic acid-lipid particles to the target tissues. Such components include, but are not limited to, PEG-lipid conjugates, such as PEG-ceramides or PEG-phospholipids (such as PEG-PE), ganglioside G_{M1}-modified lipids or ATTA-lipids to the particles. Typically, the concentration of the component in the particle will be about 1-20% and, more preferably from about 3-10%.

The pharmaceutical compositions of the present invention may be sterilized by conventional, well known sterilization techniques. Aqueous solutions can be packaged for use or filtered under aseptic conditions and lyophilized, the lyophilized preparation being combined with a sterile aqueous solution prior to administration. The compositions can contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, and calcium chloride. Additionally, the particle suspension may include lipid-protective agents which protect lipids against free-radical and lipid-peroxidative damages on storage. Lipophilic free-radical quenchers, such as alphatocopherol and water-soluble iron-specific chelators, such as ferrioxamine, are suitable.

In another example of their use, lipid-nucleic acid particles can be incorporated into a broad range of topical dosage forms including, but not limited to, gels, oils, emulsions and the like. For instance, the suspension containing the nucleic acid-lipid particles can be formulated and administered as topical creams, pastes, ointments, gels, lotions and the like.

Once formed, the serum-stable nucleic acid-lipid particles of the present invention are useful for the introduction of nucleic acids into cells. Accordingly, the present invention also provides methods for introducing a nucleic acids (e.g., a plasmid) into a cell. The methods are carried out in vitro or in vivo by first forming the particles as described above and then contacting the particles with the cells for a period of time sufficient for transfection to occur.

The nucleic acid-lipid particles of the present invention can be adsorbed to almost any cell type with which they are mixed or contacted. Once adsorbed, the particles can either be endocytosed by a portion of the cells, exchange lipids with cell membranes, or fuse with the cells. Transfer or incorporation of the nucleic acid portion of the particle can take place via any one of these pathways. In particular, when fusion takes place, the particle membrane is integrated into the cell membrane and the contents of the particle combine with the intracellular fluid.

Using the ERP assay of the present invention, the transfection efficiency of the SPLP or other lipid-based carrier system can be optimized. More particularly, the purpose of the ERP assay is to distinguish the effect of various cationic lipids and helper lipid components of SPLPs based on their relative effect on binding/uptake or fusion with/destabilization of the endosomal membrane. This assay allows one to determine quantitatively how each component of the SPLP or other lipid-based carrier system effects transfection efficacy, thereby optimizing the SPLPs or other lipid-based carrier systems. As explained herein, the Endosomal Release Parameter or, alternatively, ERP is defined as:

REPORTER GENE EXPRESSION/CELL SPLP UPTAKE/CELL

It will be readily apparent to those of skill in the art that any reporter gene (e.g., luciferase, β-galactosidase, green fluorescent protein, etc.) can be used. In addition, the lipid component (or, alternatively, any component of the SPLP or lipid-based formulation) can be labeled with any detectable label provided the does inhibit or interfere with uptake into the cell. Using the ERP assay of the present invention, one of skill in the art can assess the impact of the various lipid components (e.g., cationic lipid, non-cationic lipid, PEG-lipid derivative, PEG-DAG conjugate, ATTA-lipid derivative, calcium, CPLs, cholesterol, etc.) on cell uptake and transfection efficiencies, thereby optimizing the SPLP or other lipid-based carrier system. By comparing the ERPs for each of the various SPLPs or other lipid-based formulations, one can readily determine the optimized system, e.g., the SPLP or other lipid-based formulation that has the greatest uptake in the cell coupled with the greatest transfection efficiency.

Suitable labels for carrying out the ERP assay of the present invention include, but are not limited to, spectral labels, such as fluorescent dyes (e.g., fluorescein and derivatives, such as fluorescein isothiocyanate (FITC) and Oregon Green⁹; rhodamine and derivatives, such Texas red, tetrarhodimine isothiocynate (TRITC), etc., digoxigenin, biotin, phycoerythrin, AMCA, CyDyes⁴, and the like; radiolabels, such as ³H, ¹²⁵I, ³⁵S, ¹⁴C, ³²P, ³³P, etc.; enzymes, such as horse radish peroxidase, alkaline phosphatase, etc.; spectral colorimetric labels, such as colloidal gold or colored glass or plastic beads, such as polystyrene, polypropylene, latex, etc. The label can be coupled directly or indirectly to a component of the SPLP or other lipid-based carrier system using methods well known in the art. As indicated above, a wide variety of labels can be used, with the choice of label depending on sensitivity required, ease of conjugation with the SPLP component, stability requirements, and available instrumentation and disposal provisions.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

A. SPLPs Containing PEG-DAG Conjugates

Previous work has shown that plasmid DNA can be encapsulated in stabilized plasmid lipid particles containing the fusogenic lipid dioleoylphosphatidylethanolamine (DOPE), dioleoyldimethylammonium chloride (DODAC), and a polyethyleneglycol (PEG) coating attached to ceramides containing arachidoyl acyl groups. Diffusable PEG-ceramides confer the serum stability and long circulation lifetimes required to facilitate delivery to distal tumors upon systemic administration. The relationship between the stability of the diffusable PEG lipid and in vivo transfection activity can be established by comparing pharmacokinetic data of SPLP containing short and long acyl chain PEG-ceramides. Here we show that SPLP can be prepared using a series of PEG-diacylglycerol lipids (PEG-DAG). SPLP were prepared incorporating 10 mol percent PEG-dilaurylglycerol (C₁₂), PEG-dimyristylglycerol (C₁₄), PEG-dipalmitoylglycerol (C₁₆) or PEG-disterylglycerol (C₁₈) and evaluated for in vitro transfection activity, pharmacokinetics and the biodistribution of gene expression resulting from systemic administration in tumor bearing mice. PEG-DAG lipid containing SPLP demonstrate a similar relationship between acyl chain length and in vitro transfection activity to those containing PEG-ceramides. Shorter acyl chain anchors (dimyristyl (C₁₄) and dipalmitoyl (C₁₆)) result in SPLP particles that are less stable but have higher transfection activity in vitro than those incorporating longer acyl chain anchors (disteryl (C18)). Evaluation of the pharmacokinetics of PEG-DAG containing SPLP confirms a correlation between the stability of the PEG lipid component and the circulation lifetime of SPLP. SPLP containing PEG-dimyristylglycerol (C₁₄), PEG-dipalmitoylglycerol (C₁₆) and PEG-disterylglycerol (C18) demonstrated circulation half-lives of 0.75, 7 and 15 hours respectively. Extended circulation lifetime in turn correlates with an increase in tumor delivery and concomitant gene expression. Upon intravenous administration, PEG-disterylglycerol (C₁₈) containing SPLP bypass so-called ‘first pass’ organs, including the lung, and elicit gene expression in distal tumor tissue. The level of reporter gene expression observed in tumors represents a 100 to 1000-fold differential over that observed in any other tissue. This compares well with the behavior of SPLP containing PEG-ceramide C₂₀. The incorporation of PEG-DAG in SPLP confirms that small size, low surface charge and extended circulation lifetimes are prerequisite to the passive disease site targeting leading to accumulation of plasmid DNA and gene expression in tumors following systemic administration of non-viral transfection systems.

### MATERIALS AND METHODS

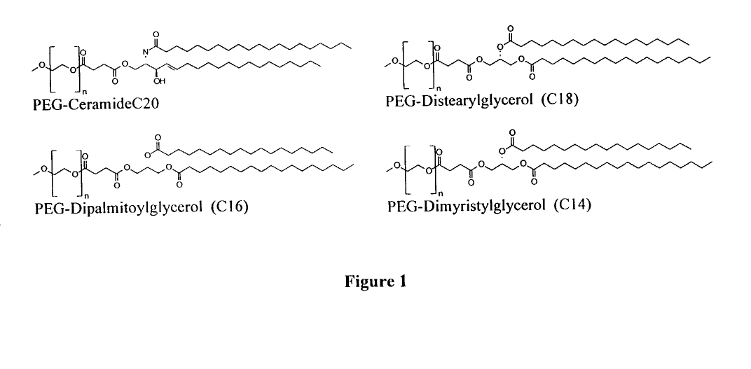

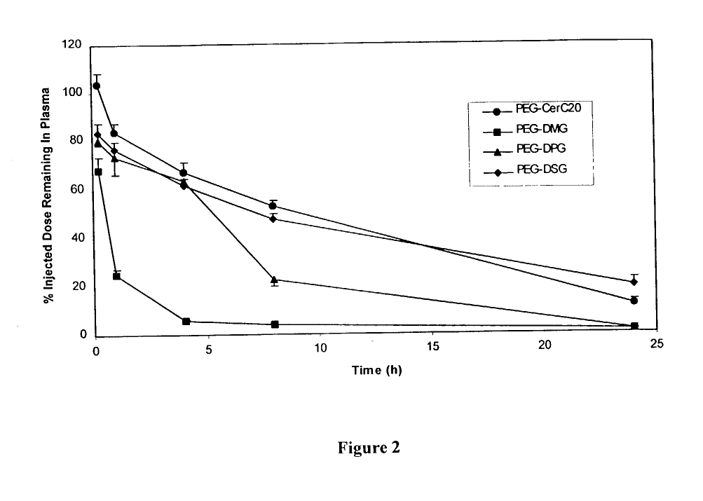

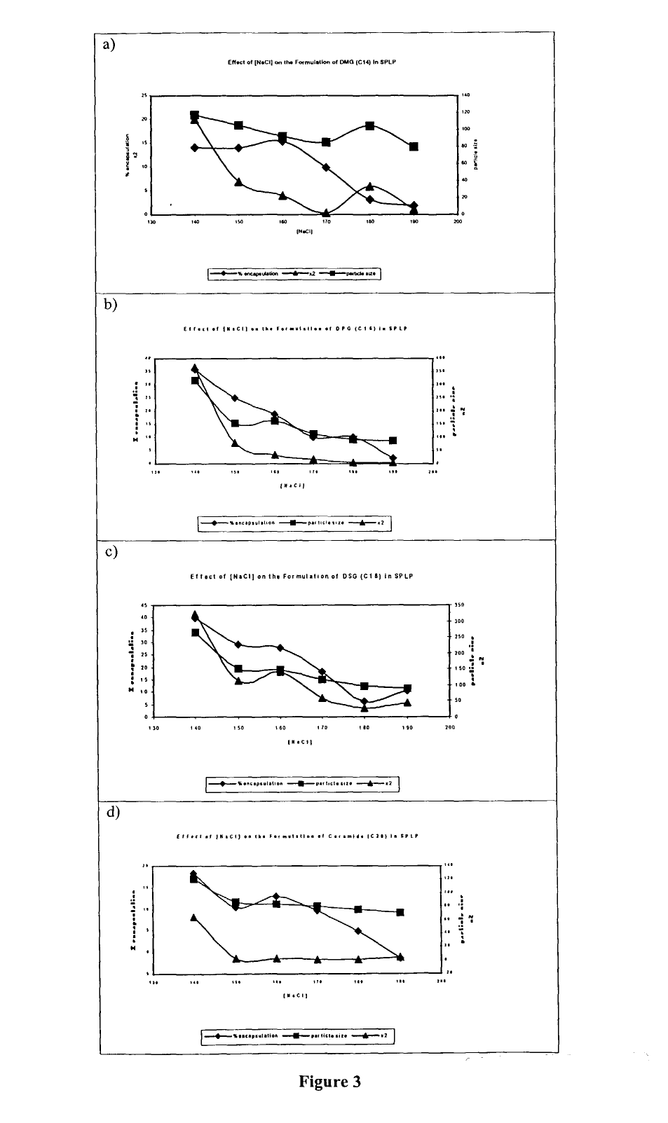

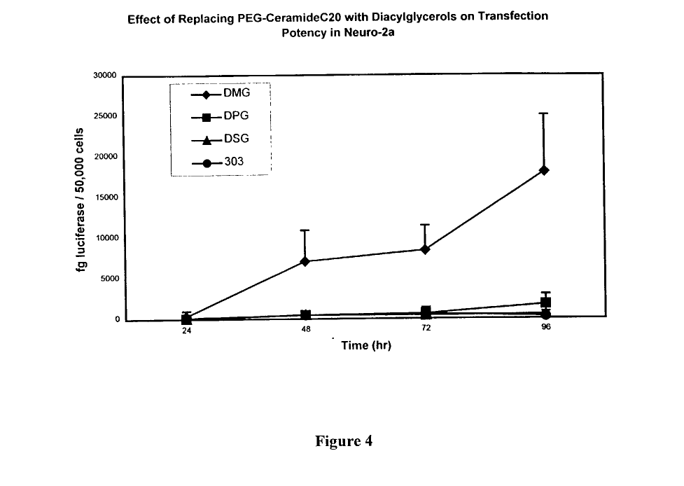

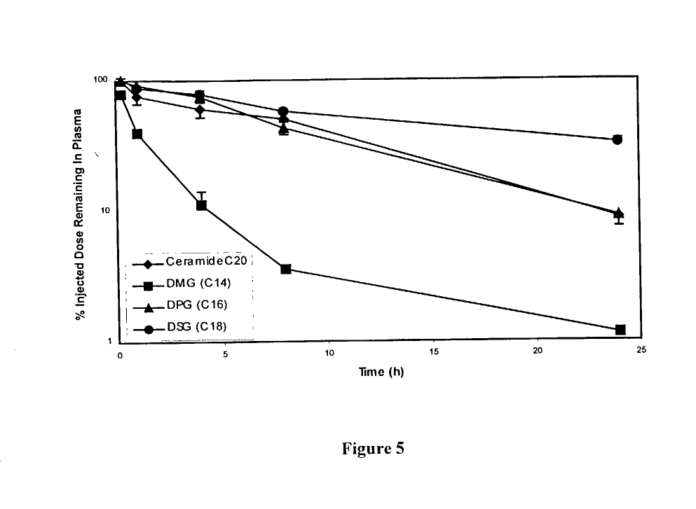

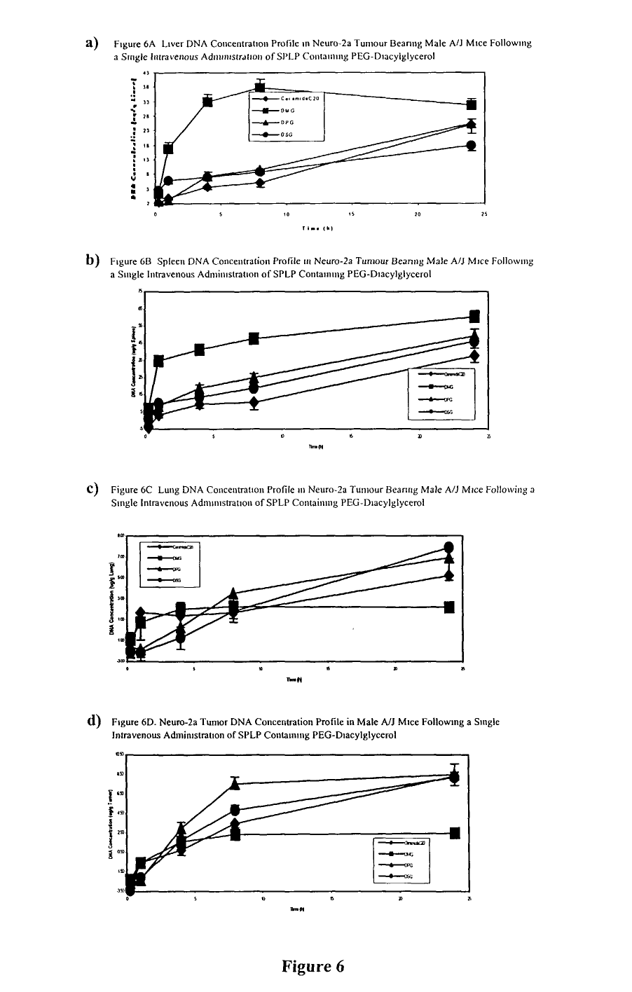

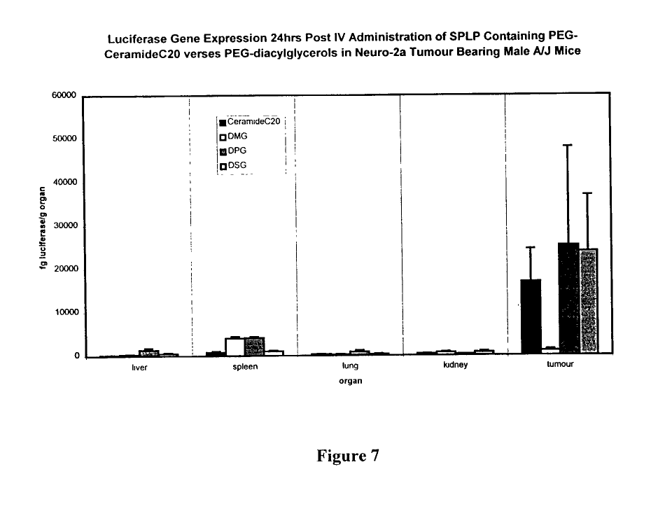

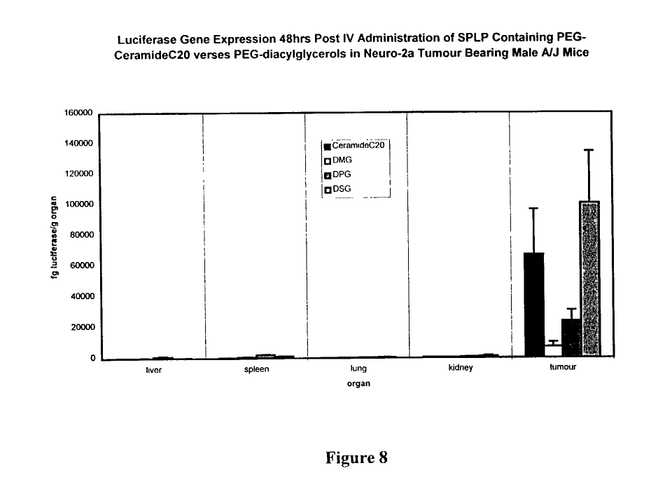

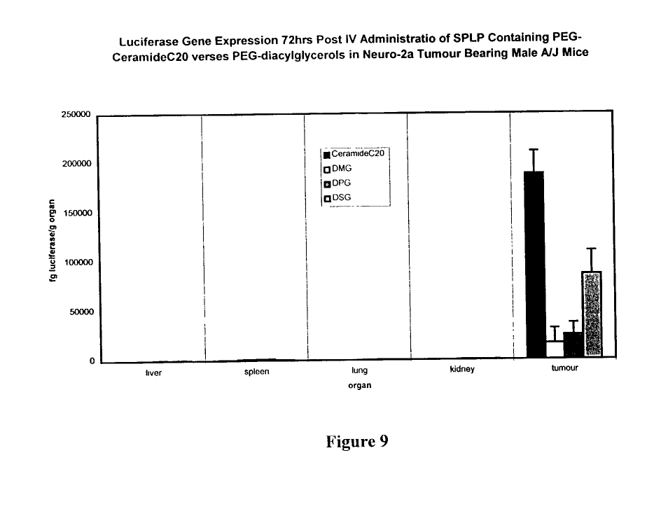

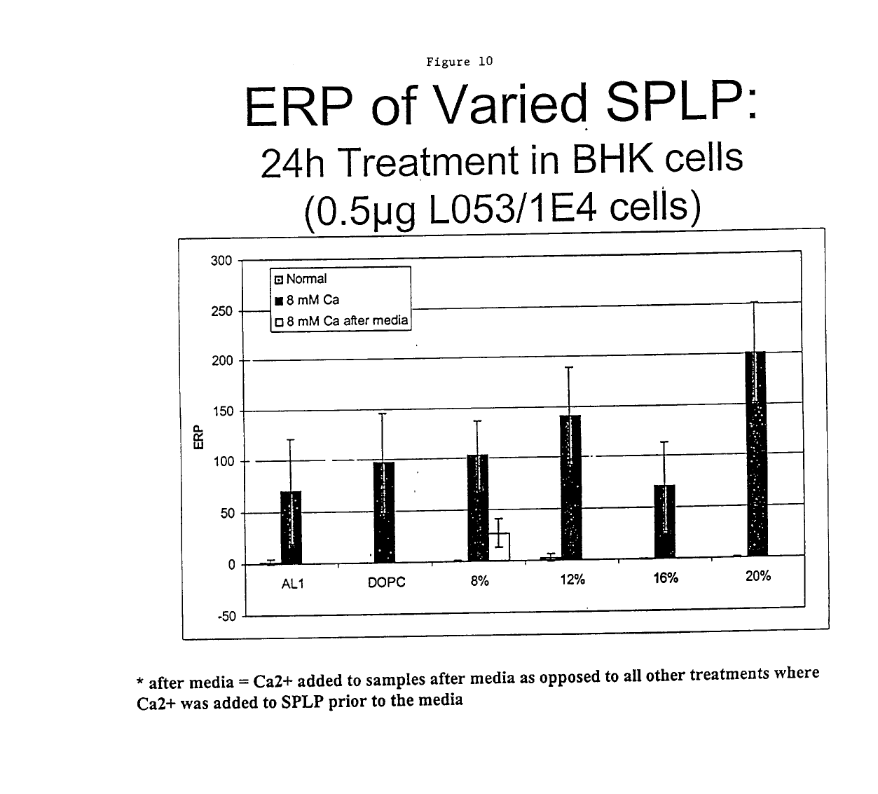

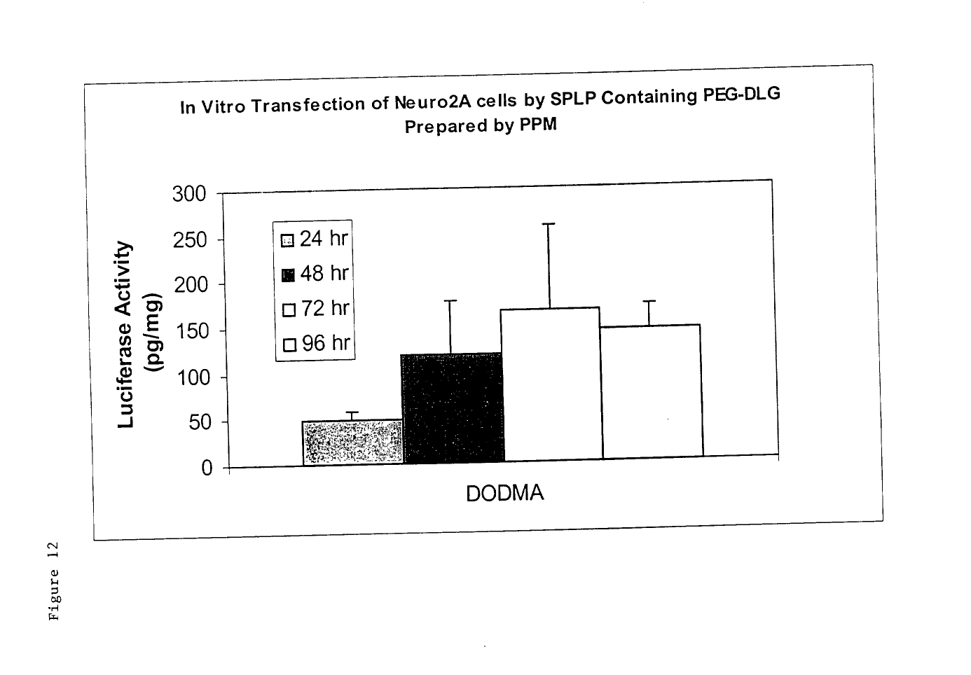

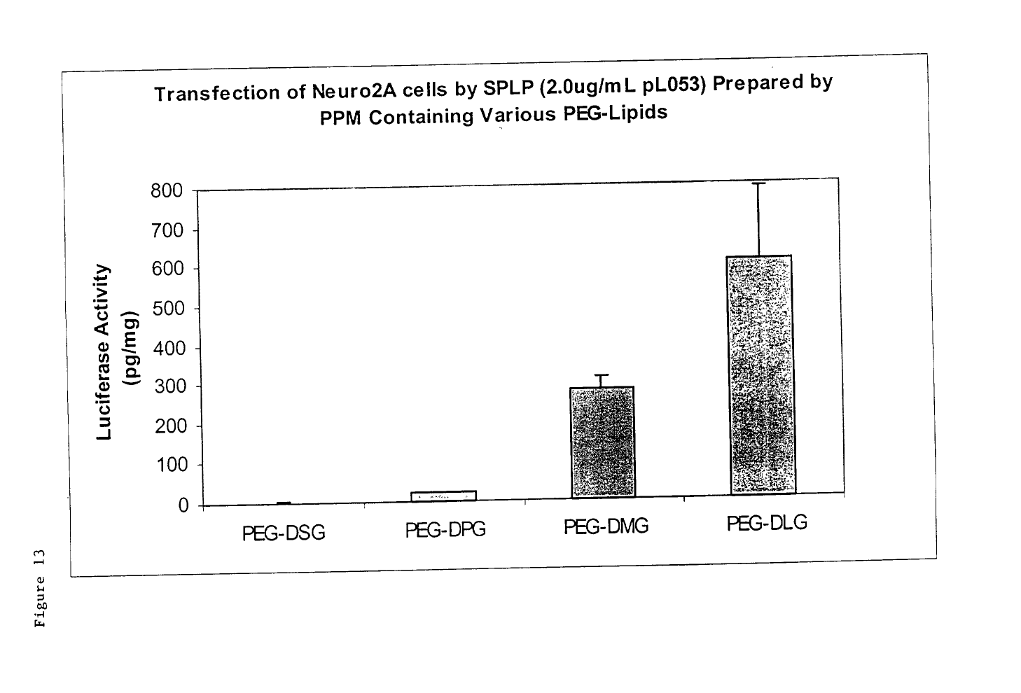

The following materials and methods were used in carrying out the experiments set forth above and in FIGS. 1-13.

Materials

DOPE and DSPC were obtained from Northern Lipids (Vancouver, BC). DODAC and the PEG-diacylglycerols were manufactured by Inex Pharmaceuticals (Burnaby, BC). The other materials, HEPES, OGP and ³H-cholesteryl hexadecyl ether, were obtained from a number of different commercial sources.

DOPE:DODAC:PEG-Diacylglycerols (82.5:7.5:10) large unilamellar vesicles were prepared via detergent dialysis in Hepes Buffered Saline (150 mM NaCl and 10 mM HEPES) for 48 hours. Lipid stock solutions were prepared in ethanol and then dried down to create a lipid film which was reconstituted in final 200 mM OGP. LUVs were labeled with ³H-cholesteryl hexadecyl ether at 1 uCi/1 mg lipid. Particle sizes were determined by nicomp analysis. Radioactivity was determined by scintillation counting with Picofluor20.

SPLP containing PEG-Diacyglycerols were formulated via detergent dialysis by varying the salt concentration to maximize the percent of DNA encapsulation. Optimal salt concentration was chosen for the 48 hour detergent dialysis. Empty vesicles were removed by one step sucrose centrifugation. 3.5% sucrose was used to separate out the empty particles from the plasmid containing PEG-Diacylglycerol formulations except for PEG-Dimyristylglycerol containing SPLP which used 5.0% sucrose. Empty vesicles migrated to the top of the tube which were fractioned out and removed.

In Vitro Transfection

5×10⁴ cells/ml were plated onto 24-well plates (1 ml). Cells were left to grow for 24 hours. 500 μl of transfection media (2.5 μg/well) was added and then incubated for stated timepoints. Transfection media was aspirated after timepoint and then exposed to complete media for another 24 hours at 37° C. in 5.0% CO₂. Complete media was removed. Cells were washed with PBS twice and stored at −70° C. until day of experiment. Cells were lysed with 150 μl of 1×CCLR containing protease inhibitors. Plates were shaken for 5 minutes. 20 μl of each sample were assayed in duplicate on a 96-well luminescence plate for luciferase activity.

Pharmacokinetics, Biodistribution, and In Vivo Gene Expression

Pharmacokinetics and biodistribution were all determined by normalizing the data to the quantity of radioactivity present. Approximately 500 μl of blood was obtained by cardiac puncture. Red blood cells and plasma were separated by centrifugation (4° C., 3000 rpm, 10 minutes) and 100 μl of plasma was used to determine radioactive counts. Organs were harvested at specified timepoints and homogenized in lysing matrix tubes (Fast Prep, 2×15 seconds, 4.5 intensity) to assay a portion of the mixture.

Gene expression was determined by luciferase assay. Organs were harvested, homogenized, and kept on ice throughout the experiment. Lysates were centrifuged (10 000 rpm, 5 minutes) and 20 μl of supernatant were assayed in duplicate on a 96-well luminescence plate for luciferase activity.

B. Optimizing Transfection Potency of SPLPs Based on the Endosomal Release Parameter (ERP)

Previously, a method to efficiently encapsulate plasmid DNA by detergent dialysis into stabilized plasmid-lipid particles (SPLP) with the lipid composition dioleoylphosphatidylethanolamine (DOPE), dioleoyldimethylammonium chloride (DODAC), and a diffusable polyethyleneglycol ceramide C₂₀ (PEG-ceramide C₂₀) (82:8: 10;mol: mol: mol), has been reported. These particles have a long half-life in circulation due to the PEG-ceramide C₂₀ and show fusogenic properties as a result of the major lipid component DOPE. Investigations have demonstrated that optimizing membrane fusion “helper lipids”, cationic lipids and the presence of calcium further enhance the transfection potency of these particles. To elucidate how these components affect the transfection process, we have analyzed their behavior based on the endosomal release parameter (ERP). The ERP represents the amount of reporter gene expression elicited by the intracellular delivery of a given amount of plasmid DNA. This parameter allows one to determine the mechanism by which changes in transfection potency occur upon modification of SPLP lipid content. Potency differences may be attributed to factors that effect cell binding and internalization. Alternately, potency differences may be the consequence of increased destabilization of the endosomal membrane in turn facilitating more efficient release of plasmid DNA into the cytosol. Determination of the endosomal release parameter has allowed us to evaluate the role of individual lipid components including cholesterol, DOPE or DSPC, and cationic lipids (e.g., DODAC, DODAP), as well as PEG-DAG conjugates in effecting the transfection process and most specifically, endosomal release. Furthermore, it has helped us understand the mechanism by which calcium plays a part in improving transfection potency. The results of these experiments may be generally applicable to the optimization of SPLP and other cationic lipid containing transfection reagents for both in vitro and in vivo applications.

Materials and Methods

The purpose of the ERP assay is to distinguish the effect of various cationic lipids and helper lipid components of SPLPs based on their relative effect on binding/uptake or fusion with/destabilization of the endosomal membrane. This assay allows one to determine quantitatively how each component effects transfection efficacy. The Endosomal Release Parameter or, alternatively, the ERP is defined as:

Reporter Gene Expression/Cell SPLP Uptake/Cell

It will be readily apparent to those of skill in the art that any reporter gene (e.g., luciferase gene, galactosidase, green fluorescent protein, etc.) can be used. In addition, the lipid component (or, alternatively, any component of the SPLP or lipid-based formulation) can be labeled with any detectable label provided the does inhibit or interfere with uptake into the cell. Using the ERP assay of the present invention, one of skill in the art can assess the impact of the various lipid components (e.g., cationic lipid, non-cationic lipid, PEG-lipid derivative, such as PEG-DAG conjugates, ATTA-lipid derivative, calcium, CPLs, cholesterol, etc.) on cell uptake and transfection efficiencies, thereby optimizing the SPLP or other lipid-based carrier system.

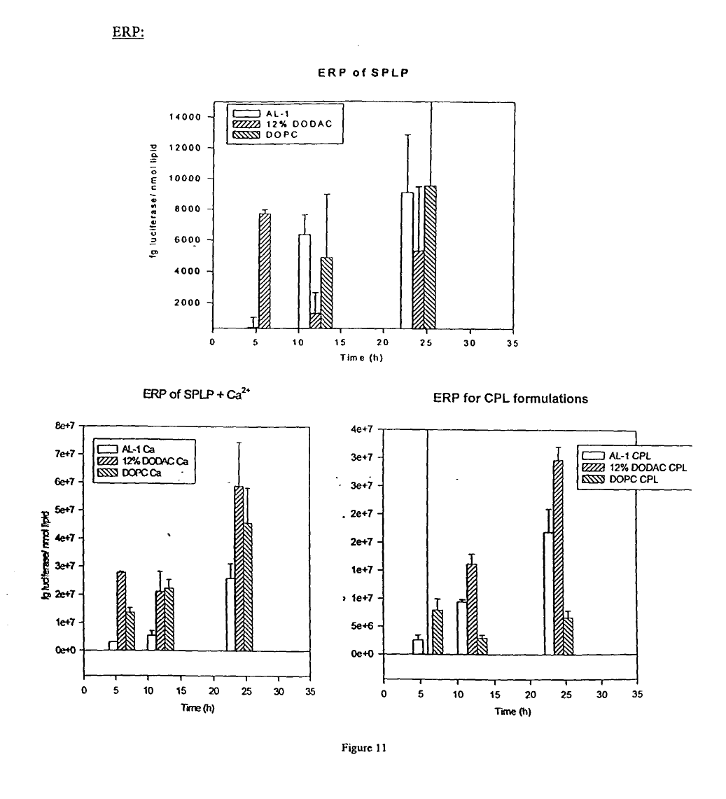

FIG. 11 illustrates the ERPs for SPLPs (A), for SPLPs plus Ca²⁺ (B) and SPLP-CPLs (C). Lipids assayed were as follows:

Titration of DODAC in the presence/absence of Ca²⁺ and CPL (ideally 8-20% DODAC, for this expt was 8 and 12% DODAC)

DOPC vs. DOPE in an 8% DODAC formulation in the presence/absence of Ca2+ and CPL

DODAC vs. AL-1 in an 8% cationic lipid formulation in the presence/absence of Ca2+ and CPL

C. Characterization of SPLPs

The SPLP method results in the encapsulation of plasmid DNA in small (diameter˜70 nm) “stabilized plasmid-lipid particles” (SPLP). SPLP consist of one plasmid per particle, encapsulated within a lipid bilayer stabilized by the presence of a poly(ethyleneglycol) (PEG) coating. SPLP exhibit extended circulation lifetimes following intravenous administration and promote delivery of intact plasmid to distal tumor sites resulting in reporter gene expression at the disease site. Here the disease site targeting and gene expression resulting from intravenous administration of SPLP in tumor bearing mice is described in detail. SPLP with long circulation times accumulate to levels corresponding to five to ten percent of the total injected dose per gram of tumor or greater than 1000 copies of plasmid DNA per cell, giving rise to levels of gene expression that are more than two orders of magnitude greater than those observed in any other tissue. Interestingly, although the liver accumulates 20-30% of the total injected dose, very low levels of gene expression are observed in the liver. This is thought to be due to the limited hepatocellular uptake of the PEG-ylated SPLP. Here we show that the in vivo transfection potential of PEG-lipid containing systems can be further enhanced through the incorporation of a cationic PEG lipid (CPL) consisting of a DSPE anchor, PEG₃₄₀₀ spacer chain and a cationic head group. When CPL are incorporated into SPLP at concentrations of 2 to 4 mol % the resulting CPL-SPLP are of a similar size and stability as native SPLP. Incorporation of CPL results in a dramatic increase in intracellular delivery and a concomitant increase in transfection activity measured both in vitro and in vivo. Specifically, CPL-SPLP yielded 10⁵-fold more in vitro gene expression than native SPLP. When CPL-SPLP are administered intravenously they yield a substantial (250 fold) increase in hepatic gene expression compared to native SPLP. The increase in CPL-SPLP potency is specific to the liver. The levels of gene expression measured in the lung, kidney, spleen or heart remain unchanged, contributing to more than two orders of magnitude differential in the gene expression measured in the liver vs. other organs. These results illustrate the potential for modulating the transfection properties of PEG-lipid containing systems while retaining the stability and small uniform size required to achieve systemic gene delivery. In particular they demonstrate that disease site targeting and tissue specific gene expression can be re-programmed by altering the lipid composition of non-viral gene delivery systems.

It is to be understood that the above description is intended to be illustrative and not restrictive. Many embodiments will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications, patents and PCT publications, are incorporated herein by reference for all purposes.

What is claimed is:

1. A nucleic acid-lipid particle, said nucleic acid-lipid particle comprising:
a nucleic acid;
a cationic lipid;
a non-cationic lipid; and
a polyethyleneglycol-diacylglycerol (PEG-DAG) conjugate.

2. The nucleic acid-lipid particle in accordance with claim 1, wherein said cationic lipid is a member selected from the group consisting of N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), and N,N-dimethyl-2,3-dioleyloxy)propylamine (DODMA), and a mixture thereof.

3. The nucleic acid-lipid particle in accordance with claim 1, wherein said non-cationic lipid is a member selected from the group consisting of dioleoylphosphatidylethanolamine (DOPE), palmitoyloleoylphosphatidylcholine (POPC), egg phosphatidylcholine (EPC), distearoylphosphatidylcholine (DSPC), cholesterol, and a mixture thereof.

4. The nucleic acid-lipid particle in accordance with claim 1, wherein said PEG-DAG conjugate is a member selected from the group consisting of PEG-dilaurylglycerol (C12), a PEG-dimyristylglycerol (C14), a PEG-dipalmitoylglycerol (C16), and a PEG-disterylglycerol (C18).

5. The nucleic acid-lipid particle in accordance with claim 1, wherein said cationic lipid comprises from about 2% to about 60% of the total lipid present in said particle.

6. The nucleic acid-lipid particle in accordance with claim 1, wherein said cationic lipid comprises from about 5% to about 45% of the total lipid present in said particle.

7. The nucleic acid-lipid particle in accordance with claim 1, wherein said cationic lipid comprises from about 5% to about 15% of the total lipid present in said particle.

8. The nucleic acid-lipid particle in accordance with claim 1, wherein said cationic lipid comprises from about 40% to about 50% of the total lipid present in said particle.

9. The nucleic acid-lipid particle in accordance with claim 1, wherein said non-cationic lipid comprises from about 5% to about 90% of the total lipid present in said particle.

10. The nucleic acid-lipid particle in accordance with claim 1, wherein said non-cationic lipid comprises from about 20% to about 85% of the total lipid present in said particle.

11. The nucleic acid-lipid particle in accordance with claim 1, wherein said PEG-DAG conjugate comprises from 1% to about 20% of the total lipid present in said particle.

12. The nucleic acid-lipid particle in accordance with claim 1, wherein said PEG-DAG conjugate comprises from 4% to about 15% of the total lipid present in said particle.

13. The nucleic acid-lipid particle in accordance with claim 1, wherein said non-cationic lipid is DSPC.

14. The nucleic acid-lipid particle in accordance with claim 13, further comprising cholesterol.

15. The nucleic acid-lipid particle in accordance with claim 14, wherein the cholesterol comprises from about 10% to about 60% of the total lipid present in said particle.

16. The nucleic acid-lipid particle in accordance with claim 14, wherein the cholesterol comprises from about 20% to about 45% of the total lipid present in said particle.

17. The nucleic acid-lipid particle in accordance with claim 1, wherein the cationic lipid comprises 7.5% of the total lipid present in said particle;
the non-cationic lipid comprises 82.5% of the total lipid present in said particle; and
the PEG-DAG conjugate comprises 10% of the total lipid present in said particle.

18. The nucleic acid-lipid particle in accordance with claim 1, wherein the nucleic acid-lipid particle comprises:
DODMA;
DSPC; and
a PEG-DAG conjugate.

19. The nucleic acid-lipid particle in accordance with claim 18, wherein the PEG-DAG conjugate is PEG-dilaurylglycerol (C12).

20. The nucleic acid-lipid particle in accordance with claim 19, further comprising cholesterol.

21. The nucleic acid-lipid particle in accordance with claim 18, wherein the PEG-DAG conjugate is PEG-dimyristylglycerol (C14).

22. The nucleic acid-lipid particle in accordance with claim 21, further comprising cholesterol.

23. The nucleic acid-lipid particle in accordance with claim 18, wherein the PEG-DAG conjugate is PEG-dipalmitoylglycerol (C16).

24. The nucleic acid-lipid particle in accordance with claim 23, further comprising cholesterol.

25. The nucleic acid-lipid particle in accordance with claim 18, wherein the PEG-DAG conjugate is PEG-disterylglycerol (C18).

26. The nucleic acid-lipid particle in accordance with claim 25, further comprising cholesterol.

27. The nucleic acid-lipid particle in accordance with claim 1, wherein said nucleic acid is DNA.

28. The nucleic acid-lipid particle in accordance with claim 1, wherein said nucleic acid is a plasmid.

29. The nucleic acid-lipid particle in accordance with claim 1, wherein said nucleic acid is an antisense oligonucleotide.

30. The nucleic acid-lipid particle in accordance with claim 1, wherein said nucleic acid is a ribozyme.

31. The nucleic acid-lipid particle in accordance with claim 1, wherein said nucleic acid encodes a therapeutic product of interest.

32. The nucleic acid-lipid particle in accordance with claim 31, wherein said therapeutic product of interest is a peptide or protein.

33. The nucleic acid-lipid particle in accordance with claim 1, wherein the nucleic acid in said nucleic acid-lipid particle is not substantially degraded after exposure of said particle to a nuclease at 37° C. for 20 minutes.

34. The nucleic acid-lipid particle in accordance with claim 1, wherein the nucleic acid in said nucleic acid-lipid particle is not substantially degraded after incubation of said particle in serum at 37° C. for 30 minutes.

35. The nucleic acid-lipid particle in accordance with claim 1, wherein the nucleic acid is fully encapsulated in said nucleic acid-lipid particle.

36. A pharmaceutical composition comprising a nucleic acid-lipid particle in accordance with claim 1 and a pharmaceutically acceptable carrier.

37. A pharmaceutical composition in accordance with claim 36 comprising a nucleic acid-lipid particle, wherein the nucleic acid-lipid particle comprises: DODMA, DSPC, and a PEG-DAG conjugate;
and a pharmaceutically acceptable carrier.

38. A pharmaceutical composition in accordance with claim 37, wherein the PEG-DAG conjugate is PEG-dilaurylglycerol (C12).

39. A pharmaceutical composition in accordance with claim 37, wherein the PEG-DAG conjugate is PEG-dimyristylglycerol (C14).

40. A pharmaceutical composition in accordance with claim 37, wherein the PEG-DAG conjugate is PEG-dipalmitoylglycerol (C16).

41. A pharmaceutical composition in accordance with claim 37, wherein the PEG-DAG conjugate is PEG-disterylglycerol (C18).

42. A method of introducing a nucleic acid into a cell; said method comprising contacting said cell with a nucleic acid-lipid particle comprising a cationic lipid, a non-cationic lipid, a PEG-DAG conjugate, and a nucleic acid.
